(19) United States
(12) Patent Application Publication
(10) Pub. No.: US 2013/0274618 A1
(43) Pub. Date: Oct. 17, 2013

(54) GUIDEWIRE SYSTEM FOR USE IN TRANSCATHETER AORTIC VALVE IMPLANTATION PROCEDURES

(71) Applicant: BOSTON SCIENTIFIC SCIMED, INC., Maple Grove, MN (US)

(72) Inventors: Dongming Hou, Tucker, GA (US); Huisun Wang, Maple Grove, MN (US); Pu Zhou, Maple Grove, MN (US); Barbara A. Huibregtse, Westborough, MA (US)

(73) Assignee: BOSTON SCIENTIFIC SCIMED, INC., Maple Grove, MN (US)

(21) Appl. No.: 13/863,935

(22) Filed: Apr. 16, 2013

Related U.S. Application Data
(60) Provisional application No. 61/625,362, filed on Apr. 17, 2012.

(51) Int. Cl. A61B 5/0215; A61F 2/24

(52) U.S. Cl. CPC A61B 5/0215; A61F 2/2427
USPC 600/486; 623/2.11

(57) ABSTRACT

A guidewire system may include a tubular guidewire including at least one lumen and a plurality of apertures disposed through an outer wall, and at least one pressure wire slidably disposed within the at least one lumen, the at least one pressure wire having at least one pressure sensor disposed thereon. A method of measuring a blood pressure gradient across a treatment site may include advancing the tubular guidewire to the treatment site, positioning at least one aperture distal and at least one aperture proximal of the treatment site, translating the pressure wire within the tubular guidewire, positioning the pressure wire such that a distal pressure sensor is disposed adjacent the at least one aperture distal of the treatment site, and a proximal pressure sensor is disposed adjacent the at least one aperture proximal of the treatment site, and measuring a blood pressure gradient across the treatment site.

### CROSS-REFERENCE TO RELATED APPLICATIONS

This application claims priority to U.S. Provisional Application No. 61/625,362 filed Apr. 17, 2012.

### TECHNICAL FIELD

The disclosure relates generally to medical devices and more particularly to medical devices that are adapted for use in procedures for repairing heart valves.

### BACKGROUND

Aortic valve stenosis is a frequent expression of valvular heart disease, and may often be a leading indicator for valve replacement therapy in Europe and the United States. The prevalence of aortic valve stenosis tends to increase in older population groups. In some cases, traditional open-heart valve replacement surgery is not suitable for patients with higher surgical risk factors. Alternate therapies, and/or linking therapies that may transition an at-risk patient to a more suitable condition for traditional open-heart valve replacement surgery, may be beneficial in improving the lifestyle of patients suffering from aortic valve stenosis.

A continuing need exists for improved devices and methods for use in alternative or predecessor treatments to traditional open-heart valve replacement surgery.

### SUMMARY

A guidewire system may include a tubular guidewire having an open proximal end, a closed distal end, and a length extending therebetween, the tubular guidewire including at least one lumen extending from the proximal end to the distal end, and at least one pressure wire slidably disposed within the at least one lumen, the at least one pressure wire having a length and at least one pressure sensor disposed thereon, wherein the tubular guidewire includes a plurality of apertures disposed through an outer wall of the tubular guidewire.

A method of measuring a blood pressure gradient across a treatment site may include obtaining a tubular guidewire system comprising a tubular guidewire having an open proximal end, a closed distal end, and a length extending therebetween, the tubular guidewire including at least one lumen extending from the proximal end to the distal end, and a pressure wire slidably disposed within the at least one lumen, the pressure wire having a length, a proximal pressure sensor disposed thereon, and a distal pressure sensor disposed thereon, wherein the tubular guidewire includes a plurality of apertures disposed through an outer wall of the tubular member. A method of measuring a blood pressure gradient across a treatment site may include advancing the distal end of the tubular guidewire upstream within a patient's vasculature to the treatment site, positioning the distal end of the tubular guidewire distal of the treatment site such that at least one of the plurality of apertures is disposed distal of the treatment site and at least one of the plurality of apertures is disposed proximal of the treatment site, translating the pressure wire longitudinally within the at least one lumen of the tubular guidewire, positioning the pressure wire within the at least one lumen such that the distal pressure sensor is disposed distal of the treatment site and adjacent the at least one of the plurality of apertures disposed distal of the treatment site, and the proximal pressure sensor is disposed proximal of the treatment site and adjacent the at least one of the plurality of apertures disposed proximal of the treatment site, and measuring a blood pressure gradient across the treatment site using the proximal pressure sensor and the distal pressure sensor.

Although discussed with specific reference to use within the coronary vasculature of a patient, for example to repair a heart valve, medical devices and methods of use in accordance with the disclosure can be adapted and configured for use in other parts of the anatomy, such as the digestive system, the respiratory system, or other parts of the anatomy of a patient.

### BRIEF DESCRIPTION OF THE FIGURES

FIG. 1 is a schematic partial cross-sectional view of an aortic heart valve having an example guidewire system disposed therein;

FIG. 1A is a schematic partial cross-sectional view of an aortic heart valve having an example guidewire system disposed therein;

FIG. 2 is a side view of an example guidewire system;

FIG. 2A is a partial cross-sectional view of a portion of the example guidewire system of FIG. 2;

FIG. 3 is a side view of an example guidewire system;

FIG. 3A is a partial cross-sectional view of a portion of the example guidewire system of FIG. 3;

FIG. 4 is a side view of an example guidewire system;

FIG. 4A is a partial cross-sectional view of a portion of the example guidewire system of FIG. 4; and

FIG. 5 is a schematic side view of an example guidewire system.

While the invention is amenable to various modifications and alternative forms, specifics thereof have been shown by way of example in the drawings and will be described in greater detail below. It should be understood, however, that the intention is not to limit the invention to the particular embodiments described. On the contrary, the intention is to cover all modifications, equivalents, and alternatives falling within the spirit and scope of the invention.

### DETAILED DESCRIPTION

For the following defined terms, these definitions shall be applied, unless a different definition is given in the claims or elsewhere in this specification.

The terms “upstream” and “downstream” refer to a position or location relative to the direction of blood flow through a particular element or location, such as a vessel (i.e., the aorta), a heart valve (i.e., the aortic valve), and the like.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the term “about” may include numbers that are rounded to the nearest significant figure.

Weight percent, percent by weight, wt %, wt-%, % by weight, and the like are synonyms that refer to the concentration of a substance as the weight of that substance divided by the weight of the composition and multiplied by 100.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following description should be read with reference to the drawings wherein like reference numerals indicate like elements throughout the several views. One of ordinary skill in the art will readily appreciate and understand that a particular element or feature from any disclosed or illustrated example embodiment herein may be incorporated into any other example embodiment unless expressly stated otherwise. The detailed description and drawings are intended to illustrate but not limit the claimed invention.

Diseases and/or medical conditions that impact the cardiovascular system are prevalent in the United States and throughout the world. Traditionally, treatment of the cardiovascular system was often conducted by directly accessing the impacted part of the system. For example, treatment of a blockage in one or more of the coronary arteries was traditionally treated using coronary artery bypass surgery. As can be readily appreciated, such therapies are rather invasive to the patient and require significant recovery times and/or treatments. More recently, less invasive therapies have been developed, for example, where a blocked coronary artery could be accessed and treated via a percutaneous catheter (e.g., angioplasty). Such therapies have gained wide acceptance among patients and clinicians.

Some relatively common medical conditions may include or be the result of inefficiency, ineffectiveness, or complete failure of one or more of the valves within the heart. For example, failure of the aortic valve can have a serious effect on a human and could lead to a serious health condition and/or death if not dealt with. A human heart includes several different heart valves, including aortic, pulmonary, mitral, and tricuspid valves, which control the flow of blood to and from the heart. Over time, a heart valve may become obstructed, narrowed, and/or less flexible (i.e., stenosed) due to hardening, calcium deposition, or other factors, thereby reducing the flow of blood through the valve and/or increasing the pressure within the chambers of the heart as the heart attempts to pump the blood through the vasculature. One traditional treatment method is valve replacement, where the stenosed valve is removed and a replacement tissue or mechanical valve is implanted via open heart surgery. Alternative treatments, including percutaneous valve replacement procedures (i.e., transcatheter aortic valve implantation, or TAVI) which may deliver and implant a replacement heart valve (i.e., aortic valve), have been developed which may be much less invasive to the patient. The devices and methods described herein may provide additional desirable features and benefits for use in such procedures.

A typical aortic valve may comprise three leaflets, although two leaflet and four leaflet valves are known to occur in a portion of the population. For simplicity, the following discussion will be described in the context of treating a typical aortic valve. However, it is fully contemplated that the devices and methods described herein may be adapted for use in the treatment of a two or four (or more) leaflet heart valve and/or a non-aortic heart valve. One of ordinary skill in the art will understand that in the event of treating a non-aortic heart valve, the relative orientations and directions associated with the described devices and methods may be modified to accommodate the specifics (i.e., orientation, location, size, etc.) of the heart valve undergoing treatment.

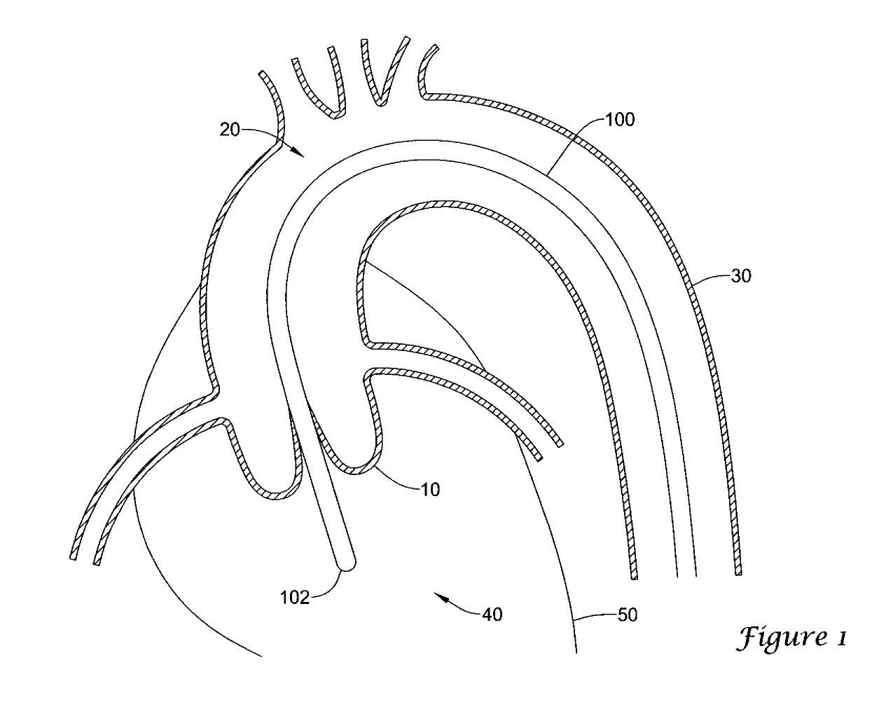

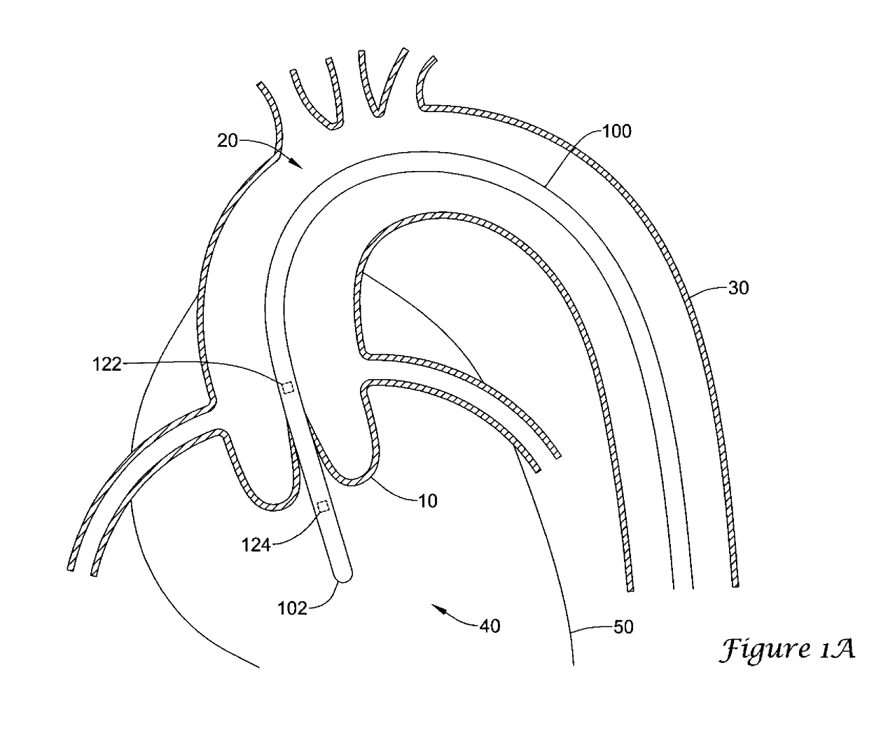

FIG. 1 illustrates an example TAVI guidewire 100 positioned within an aortic valve 10. As shown in FIG. 1, the TAVI guidewire 100 may extend upstream through the aorta 30, across or through the aortic arch 20, and through the aortic valve 10 into the left ventricle 40 of a patient's heart 50. In some embodiments, a distal end 102 of the TAVI guidewire 100 may be positioned within the left ventricle 40 during a TAVI procedure.

In some embodiments, the TAVI guidewire 100 may include a proximal pressure sensor 122 and a distal pressure sensor 124. The proximal pressure sensor 122 and the distal pressure sensor 124 may be longitudinally spaced apart along a length of the TAVI guidewire 100, as shown in FIG. 1A. In some embodiments, the TAVI guidewire 100 may include less than two pressure sensors or more than two pressure sensors as appropriate for the procedure being performed. In some embodiments, the distal pressure sensor 124 may be disposed adjacent the distal end 102 of the TAVI guidewire 100, and the distal pressure sensor 124 may be positioned distal of a treatment site (i.e., a patient's aortic valve 10) or within the left ventricle 40, during a TAVI procedure, for example. In some embodiments, the proximal pressure sensor 122 may be spaced apart proximally from the distal pressure sensor 124, and the proximal pressure sensor 122 may be positioned proximal of a treatment site (i.e., a patient's aortic valve 10), or within the aortic arch 20, during a TAVI procedure, for example.

Positioning the distal pressure sensor 124 distal of the treatment site (i.e., the patient's aortic valve 10), or within the left ventricle 40, and the proximal pressure sensor 122 proximal of the treatment site (i.e., the patient's aortic valve 10), or within the aortic arch 20, as seen in FIG. 1A, may permit a practitioner to measure and/or track blood pressure within the left ventricle 40, blood pressure within the aortic arch 20, and/or a blood pressure gradient across the treatment site (i.e., the patient's aortic valve 10), or a difference in blood pressure measured upstream of the treatment site (i.e., the patient's aortic valve 10) relative to blood pressure measured downstream of the treatment site (i.e., the patient's aortic valve 10), before, during, and/or after a TAVI procedure (i.e., percutaneous implantation of a replacement heart valve within the existing aortic valve 10). Blood pressure within the left ventricle 40 can be an indirect indicator of valve (i.e., aortic valve 10) function. A preferred blood pressure gradient is as low or as small as possible. In other words, the blood pressure measured within the left ventricle 40 and the blood pressure measured within the aortic arch 20 are very close in value or have very little difference in value (i.e., within about 25%, within about 15%, within about 10%, within about 5%, or less than 5% difference).

In some embodiments, the TAVI guidewire may be tubular or hollow in construction, with one or more lumens disposed therein, such as, for example, a hypotube or a thin-walled tubular catheter. Those of skill in the art and others will recognize that the materials, structures, and dimensions of the TAVI guidewire are dictated primarily by the desired characteristics and function of the final guidewire, and that any of a broad range of materials, structures, and dimensions can be used.

For example, the TAVI guidewire may be formed of any materials suitable for use, dependent upon the desired properties of the TAVI guidewire. Some examples of suitable materials include metals, metal alloys, polymers, composites, or the like, or combinations or mixtures thereof. Some examples of suitable metals and metal alloys include stainless steel, such as 304V, 304L, and 316L stainless steel; alloys including nickel-titanium alloy such as linear elastic or superelastic (i.e., pseudoelastic) nitinol; nickel-chromium alloy; nickel-chromium-iron alloy; cobalt alloy; tungsten or tungsten alloys; MP35-N (having a composition of about 35% Ni, 35% Co, 20% Cr, 9.75% Mo, a maximum 1% Fe, a maximum 1% Ti, a maximum 0.25% C, a maximum 0.15% Mn, and a maximum 0.15% Si); hastelloy; monel 400; inconel 625; or the like; or other suitable material, or combinations or alloys thereof. In some embodiments, it is desirable to use metals or metal alloys that are suitable for metal joining techniques such as welding, soldering, brazing, crimping, friction fitting, adhesive bonding, etc. The particular material used can also be chosen in part based on the desired flexibility requirements or other desired characteristics.

The word nitinol was coined by a group of researchers at the United States Naval Ordinance Laboratory (NOL) who were the first to observe the shape memory behavior of this material. The word nitinol is an acronym including the chemical symbol for nickel (Ni), the chemical symbol for titanium (Ti), and an acronym identifying the Naval Ordinance Laboratory (NOL).

Within the family of commercially available nitinol alloys is a category designated “linear elastic” which, although similar in chemistry to conventional shape memory and superelastic (i.e., pseudoelastic) varieties, exhibits distinct and useful mechanical properties. By skilled applications of cold work, directional stress and heat treatment, the wire is fabricated in such a way that it does not display a substantial “superelastic plateau” or “flag region” in its stress/strain curve. Instead, as recoverable strain increases, the stress continues to increase in an essentially linear relationship until plastic deformation begins. In some embodiments, the linear elastic nickel-titanium alloy is an alloy that does not show any martensite/austenite phase changes that are detectable by DSC and DMTA analysis over a large temperature range.

For example, in some embodiments, there are no martensite/austenite phase changes detectable by DSC and DMTA analysis in the range of about −60° C. to about 120° C. The mechanical bending properties of such a material are therefore generally inert to the effect of temperature over this very broad range of temperatures. In some particular embodiments, the mechanical properties of the alloy at ambient or room temperature are substantially the same as the mechanical properties at body temperature. In some embodiments, the use of the linear elastic nickel-titanium alloy allows the guidewire to exhibit superior “pushability” around tortuous anatomy.

In some embodiments, the linear elastic nickel-titanium alloy is in the range of about 50 to about 60 weight percent nickel, with the remainder being essentially titanium. In some particular embodiments, the composition is in the range of about 54 to about 57 weight percent nickel. One example of a suitable nickel-titanium alloy is FHP-NT alloy commercially available from Furukawa Techno Material Co. of Kanagawa, Japan. Some examples of suitable nickel-titanium alloys include those disclosed in U.S. Pat. Nos. 5,238,004 and 6,508,803, which are incorporated herein by reference. In some other embodiments, a superelastic alloy, for example a superelastic nitinol, can be used to achieve desired properties.

Portions or all of the TAVI guidewire, or other structures (i.e., markers, for example) included within the TAVI guidewire, may in some cases be doped with, coated or plated with, made of, or otherwise include a radiopaque material. Radiopaque materials are understood to be materials capable of producing a relatively bright image on a fluoroscopy screen or another imaging technique during a medical procedure. This relatively bright image aids the user of the TAVI guidewire in determining its location. Some examples of radiopaque materials can include, but are not limited to, gold, platinum, palladium, tantalum, tungsten alloy, polymer material loaded with a radiopaque filler, and the like, or combinations or alloys thereof.

Additionally, in some instances a degree of MRI compatibility can be imparted into the TAVI guidewire. For example, to enhance compatibility with Magnetic Resonance Imaging (MRI) machines, the TAVI guidewire, or other portions of the TAVI guidewire, can be made in a manner that would impart a degree of MRI compatibility. For example, the TAVI guidewire, or portions thereof, may be made of a material that does not substantially distort the image and create substantial artifacts (artifacts are gaps in the image) during MRI imaging. Certain ferromagnetic materials, for example, may not be suitable because they may create artifacts in an MRI image. The TAVI guidewire, or portions thereof, may also be made from a material that the MRI machine can image. Some materials that exhibit these characteristics include, for example, tungsten, Elgiloy™, MP35N, nitinol, and the like, and others, or combinations or alloys thereof.

As indicated above, the TAVI guidewire may generally have a tubular construction with a hollow cross-section defined by at least one wall defining at least one lumen extending therein. In some embodiments, each of the one or more lumens may be adapted and/or configured to house or surround at least a portion of an example pressure wire, as will be described in more detail below. The particular cross-sectional shape of the TAVI guidewire can be any desired shape, for example rounded, oval, rectangular, square, polygonal, and the like, or other such various cross-sectional geometries. The cross-sectional geometries along the length of the TAVI guidewire can be constant or can vary. For example, the figures depict the TAVI guidewire as having a generally constant round cross-sectional shape, but it can be appreciated that other cross-sectional shapes or combinations of shapes, while not expressly illustrated, may be utilized without departing from the spirit of the invention.

Additionally, the TAVI guidewire may include one or more tapers or tapered regions, and one or more constant diameter sections, or may generally include a constant inner and outer diameter. The tapers and/or constant diameters may be manifested in variations and/or consistencies in the size of the outer diameter, inner diameter, and/or wall thickness of the TAVI guidewire. Any tapered regions may be linearly tapered, tapered in a curvilinear fashion, uniformly tapered, non-uniformly tapered, or tapered in a step-wise fashion. The angle of any such tapers can vary, depending upon the desired flexibility characteristics. The length of the taper may be selected to obtain a more (longer length) or less (shorter length) gradual transition in stiffness/flexibility characteristics. It can be appreciated that essentially any portion of the TAVI guidewire may be tapered or can have a constant diameter, and that any tapers and/or constant diameter can extend in either the proximal or the distal direction, for example, to achieve the desired flexibility, stiffness, and/or torque transmission characteristics.

In some embodiments, the TAVI guidewire can have one or more lumens having an inner diameter that is in the range of about 0.008 inch to about 0.060 inch in size, and in some embodiments, in the range of about 0.015 inch to about 0.030 inch in size. Additionally, in some embodiments, the TAVI guidewire can have an outer diameter that is in the range of about 0.010 inch to about 0.070 in size, and in some embodiments, in the range of about 0.030 inch to about 0.040 inch in size, and in some embodiments, about 0.035 inch. It should be understood however, that these and other dimensions provided herein are by way of example embodiments only, and that in other embodiments, the size of the inner and outer diameter of the TAVI guidewire can vary greatly from the dimensions given, depending upon the desired characteristics and function of the device.

The outer diameter of the TAVI guidewire, including any tapered and/or constant diameter portions, may be formed by any one of a number of different techniques, for example, by centerless grinding methods, stamping methods, extrusion methods, co-extrusion methods, and the like. A centerless grinding technique may utilize an indexing system employing sensors (e.g., optical/reflective, magnetic) to avoid excessive grinding. In addition, the centerless grinding technique may utilize a CBN or diamond abrasive grinding wheel that is well shaped and dressed to avoid grabbing the TAVI guidewire during the grinding process. In some embodiments, centerless grinding can be achieved using a Royal Master HI-AC centerless grinder. Some examples of suitable grinding methods are disclosed in U.S. patent application Ser. No. 10/346,698 filed Jan. 17, 2003 (Pub. No. U.S. 2004/0142643), which is herein incorporated by reference.

The TAVI guidewire can also include structure or otherwise be adapted and/or configured to achieve a desired level of stiffness, torqueability, flexibility, and/or other characteristics. The desired stiffness, torqueability, lateral flexibility, bendability or other such characteristics of the TAVI guidewire can be imparted, enhanced, or modified by the particular structure that may be used or incorporated into the TAVI guidewire. As can thus be appreciated, the flexibility of the tubular member can vary along its length, for example, such that the flexibility can be higher at the distal end relative to the proximal end, or vice versa. However, in some embodiments, the TAVI guidewire can have a substantially constant flexibility along the entire length thereof.

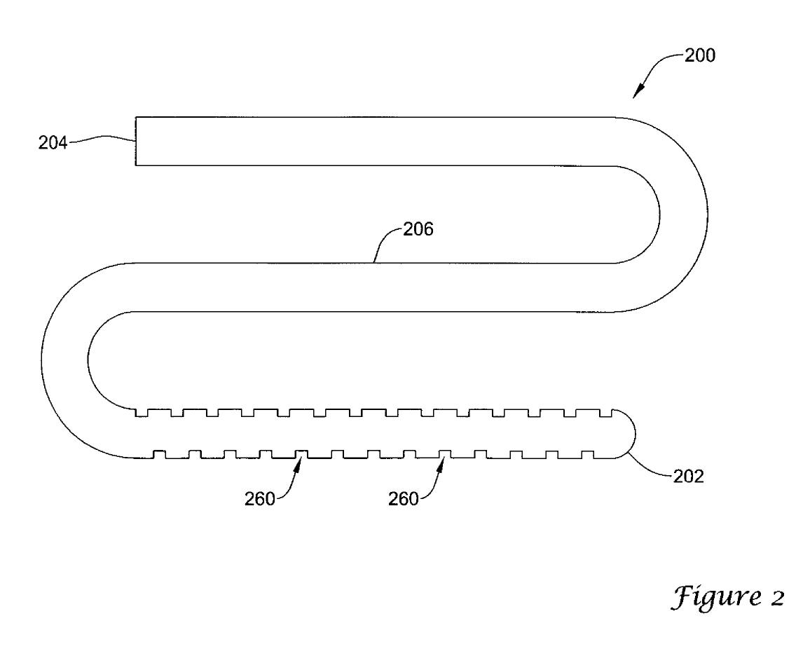

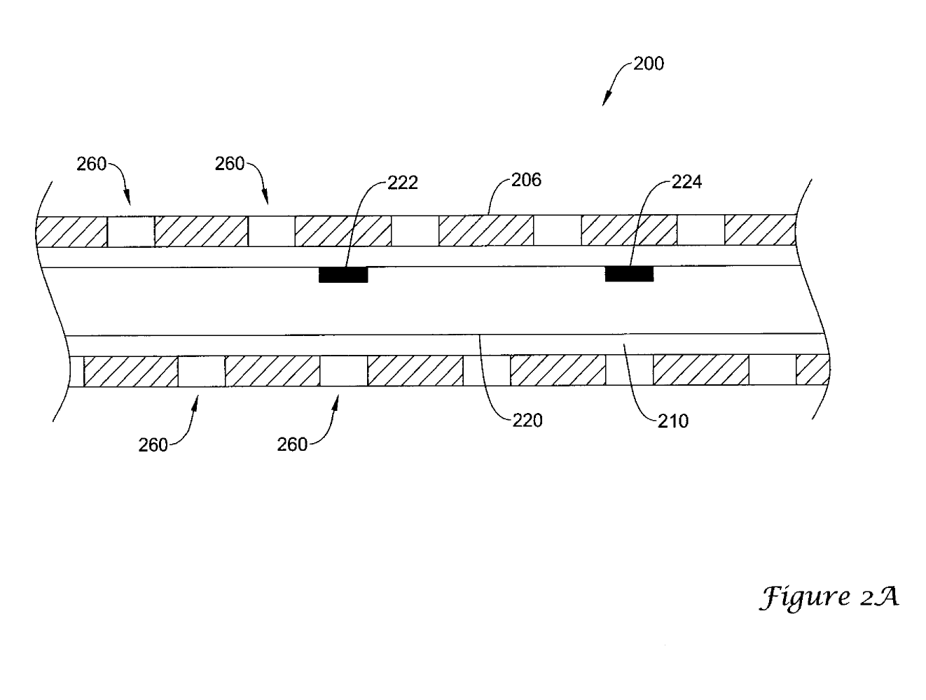

One manner of imparting additional flexibility is to selectively remove material from portions of the TAVI guidewire. For example, with reference to FIGS. 2 and 2A, a TAVI guidewire 200 may include a thin wall tubular structure including a plurality of apertures 260, such as grooves, cuts, slits, slots, or the like, formed in a portion of, or along the entire length of, the TAVI guidewire 200. The plurality of apertures 260 may be formed such that one or more spines or beams are formed in the TAVI guidewire 200. Such spines or beams could include portions of the TAVI guidewire 200 that remain after the plurality of apertures 260 is formed in the thin wall tubular structure of the TAVI guidewire 200, and may act to maintain a relatively high degree of torsional stiffness while maintaining a desired level of lateral flexibility due to the plurality of apertures 260. Such structure may be desirable because it may allow TAVI guidewire 200, or portions thereof, to have a desired level of laterally flexibility as well as have the ability to transmit torque and pushing forces from the proximal end 204 to the distal end 202. The plurality of apertures 260 can be formed in essentially any known way. For example, the plurality of apertures 260 can be formed by methods such as micro-machining, saw-cutting, laser cutting, grinding, milling, casting, molding, chemically etching or treating, or other known methods, and the like. In some such embodiments, the structure of the TAVI guidewire 200 is formed by cutting and/or removing portions of the thin wall tubular structure to form the plurality of apertures 260.

In some embodiments, the plurality of apertures 260 can completely penetrate an outer wall 206 of the TAVI guidewire 200 such that there is fluid communication between a lumen 210 extending therethrough (i.e., defined by the outer wall 206) and an exterior of the TAVI guidewire 200 through the plurality of apertures 260. The shape and size of the plurality of apertures 260 can vary, for example, to achieve the desired characteristics. For example, the shape of the plurality of apertures 260 can vary to include essentially any appropriate shape, such as squared, round, rectangular, pill-shaped, oval, polygonal, elongated, irregular, spiral (which may or may not vary in pitch), or other suitable means or the like, and may include rounded or squared edges, and can be variable in length and width, and the like. In some embodiments, a TAVI guidewire may include a helical coil having adjacent turns spaced apart to form a plurality of apertures extending through to an interior lumen. Other configurations, arrangements, and/or combinations thereof may also be used.

In some embodiments, some adjacent apertures can be formed such that they include portions that overlap with each other about the circumference of the TAVI guidewire 200. In other embodiments, some adjacent apertures can be disposed such that they do not necessarily overlap with each other, but are disposed in a pattern that provides the desired degree of lateral flexibility. Additionally, the apertures can be arranged along the length of, or about the circumference of, the TAVI guidewire 200 to achieve desired properties. For example, the apertures can be arranged in a symmetrical pattern, such as being disposed essentially equally on opposite sides about the circumference of the TAVI guidewire 200, or equally spaced along the length of the TAVI guidewire 200, or can be arranged in an increasing or decreasing density pattern, or can be arranged in a non-symmetric or irregular pattern.

As can be appreciated, the spacing, arrangement, and/or orientation of the plurality of apertures 260, or in the associated spines or beams that may be formed, can be varied to achieve the desired characteristics. For example, the number, proximity (to one another), density, size, shape and/or depth of the plurality of apertures 260 along the length of the TAVI guidewire 200 may vary in either a stepwise fashion or consistently, depending upon the desired characteristics. For example, the quantity or proximity of the plurality of apertures 260 to one another near one end of the TAVI guidewire 200 may be high, while the quantity or proximity of the plurality of apertures 260 to one another near the other end of the TAVI guidewire 200, may be relatively low, or vice versa. For example, in the some embodiments, a distal region of the TAVI guidewire 200 may include a greater density of apertures, while a proximal region of the TAVI guidewire 200 may include a lesser density of apertures, or may even be devoid of any apertures. As such, the distal region may have a greater degree of lateral flexibility relative to the proximal region. It should be understood that similar variations in the size, shape and/or depth of the plurality of apertures 260 along the length of the TAVI guidewire 200 can also be used to achieve desired flexibility differences thereof.

In the embodiment shown in FIGS. 2 and 2A, the plurality of apertures 260 and the associated spines or beams are disposed in a generally uniform pattern along a distal portion of the TAVI guidewire 200. In this embodiment, the plurality of apertures 260 each have a length and a width, and the length of each of the plurality of apertures 260 extends generally perpendicular to the longitudinal axis of the TAVI guidewire 200. In other words, the plurality of apertures 260 may have a major axis extending along their length that extends radially about the longitudinal axis of the TAVI guidewire 200, and the major axis is generally perpendicular to the longitudinal axis of the TAVI guidewire 200.

Additionally, as seen in FIGS. 2 and 2A, the plurality of apertures 260 may be formed in alternating groups of two, wherein each of the two apertures in a group is disposed at a different longitudinal point along the length of the TAVI guidewire 200, and on an opposite side of the TAVI guidewire 200 about the circumference thereof. In some embodiments, two apertures may form a pair that is disposed at a similar longitudinal point along the length of the tubular member, and are formed on opposite sides of the TAVI guidewire 200 about the circumference thereof, along a plane substantially perpendicular to the longitudinal axis of the TAVI guidewire 200. It should be understood, however, that in other embodiments the arrangement of the apertures can be varied to achieve the desired characteristics along the length of a TAVI guidewire. For example, instead of pairs, only a single aperture, or more than two apertures, may be located at certain points along the length of the device. Additionally, the major axis of the apertures may be disposed at different angles, not necessarily perpendicular to the longitudinal axis of the TAVI guidewire.

Collectively, these Figures and this Description illustrate that changes in the arrangement, number, and configuration of a plurality of apertures may vary without departing from the scope of the invention. Some additional examples of arrangements of apertures, such as cuts or slots, formed in a tubular body are disclosed in U.S. Pat. No. 6,428,489, and in U.S. Pat. No. 6,579,246, both of which are incorporated herein by reference. Also, some additional examples of arrangements of cuts or slots formed in a tubular body for use in a medical device are disclosed in a U.S. patent application Ser. No. 10/375,493 filed Feb. 28, 2003 (Pub. No. U.S. 2004/0167437), which is incorporated herein by reference.

The flexibility characteristics of a TAVI guidewire could also be achieved using other methods, such as by the addition of material and/or one or more reinforcement members to certain portions of the TAVI guidewire. As understood by one of skill in the art, any of a broad variety of attachment techniques and/or structures can be used to attachment additional material and/or one or more reinforcement members to a TAVI guidewire. Some examples of suitable attachment techniques include welding, soldering, brazing, crimping, friction fitting, adhesive bonding, mechanical interlocking and the like.

Some examples of welding processes that can be suitable in some embodiments include LASER welding, resistance welding, TTG welding, microplasma welding, electron beam welding, friction welding, inertia welding, or the like. LASER welding equipment which may be suitable in some applications is commercially available from Unitek Miyachi of Monrovia, Calif. and Rofin-Sinar Incorporated of Plymouth, Mich. Resistance welding equipment which may be suitable in some applications is commercially available from Palomar Products Incorporated of Carlsbad, Calif. and Polaris Electronics of Olathe, Kans. TIG welding equipment which may be suitable in some applications is commercially available from Weldlogic Incorporated of Newbury Park, Calif. Microplasma welding equipment which may be suitable in some applications is commercially available from Process Welding Systems Incorporated of Smyrna, Tenn.

In some embodiments, LASER or plasma welding can be used to achieve the attachment. In LASER welding, a light beam is used to supply the necessary heat. LASER welding can be beneficial in the processes contemplated by the invention, as the use of a LASER light heat source can provide significant accuracy. It should also be understood that such LASER welding can also be used to attach other components to the device. Additionally, in some embodiments, LASER energy can be used as the heat source for soldering, brazing, or the like for attaching different components or structures of the guidewire together. Again, the use of a LASER as a heat source for such connection techniques can be beneficial, as the use of a LASER light heat source can provide substantial accuracy. One particular example of such a technique includes LASER diode soldering.

Additionally, in some other example embodiments, attachment may be achieved and/or aided through the use of a mechanical connector or body, and/or by an expandable alloy, for example, a bismuth alloy. Some examples of methods, techniques and structures that can be used to interconnect different portions of a guidewire using such expandable material are disclosed in a U.S. patent application Ser. No. 10/375,766 filed Feb. 26, 2003 (Pub. No. U.S. 2004/0167441), which is hereby incorporated herein by reference. Some methods and structures that can be used to interconnect different sections are disclosed in U.S. Pat. No. 6,918,882, and U.S. patent application Ser. No. 10/086,992 filed Feb. 28, 2002 (Pub. No. U.S. 2003/0069521), which are incorporated herein by reference.

Additionally, in some embodiments, a coating, for example a lubricious (i.e., hydrophilic, hydrophobic, etc.) or other type of coating may be applied over portions or all of the TAVI guidewire discussed above. Hydrophobic coatings such as fluoropolymers, silicones, and the like provide a dry lubricity which improves guidewire handling and device exchanges. Lubricious coatings improve steerability and improve lesion crossing capability. Suitable lubricious polymers are well known in the art and may include (but are not limited to) hydrophilic polymers such as polyarylene oxides, polyvinylpyrolidones, polyvinylalcohols, hydroxy alkyl cellulosics, algins, saccharides, caprolactones, and the like, and mixtures and combinations thereof. Hydrophilic polymers may be blended among themselves or with formulated amounts of water insoluble compounds (including some polymers) to yield coatings with suitable lubricity, bonding, and solubility. Some other examples of such coatings and materials and methods used to create such coatings can be found in U.S. Pat. Nos. 6,139,510 and 5,772,609, which are incorporated herein by reference. In some embodiments, a more distal portion of a TAVI guidewire is coated with a hydrophilic polymer, and a more proximal portion is coated with a fluoropolymer, such as polytetrafluoroethylene (PTFE).

The use of a coating layer in some embodiments can impart a desired flexibility to the TAVI guidewire. Choice of coating materials may vary, depending upon the desired characteristics. For example, coatings with a low durometer or hardness may have very little effect on the overall flexibility of the TAVI guidewire. Conversely, coatings with a high durometer may make for a stiffer and/or less flexible shaft.

FIGS. 2 and 2A illustrate an example TAVI guidewire 200 having a lumen 210 disposed therein and a plurality of apertures 260 through an outer wall 206 of the TAVI guidewire 200 disposed adjacent the distal end 202. At least some of the plurality of apertures 260 may be longitudinally spaced apart along a length of the TAVI guidewire 200. The TAVI guidewire 200 may include a pressure wire 220 slidably disposed within the lumen 210. The pressure wire 220 may include a proximal pressure sensor 222 and a distal pressure sensor 224 disposed thereon. The proximal pressure sensor 222 and the distal pressure sensor 224 may be longitudinally spaced apart along a length of the pressure wire 220. Preferably, the proximal pressure sensor 222 and the distal pressure sensor 224 are longitudinally spaced apart far enough to permit placement on opposing sides (i.e., proximal and distal) of a treatment site (i.e., a patient's aortic valve 10). The proximal pressure sensor 222 and the distal pressure sensor 224 may be adapted and/or configured to measure a blood pressure gradient across a treatment site (i.e., a patient's aortic valve 10). In some embodiments, the pressure wire 220 may be a solid wire having an outer diameter of about 0.005 inch to about 0.025 inch, or in some embodiments, about 0.014 inch. The lumen 210 of the TAVI guidewire 200 may have a diameter slightly larger than the outer diameter of the pressure wire 220.

Additionally, as the pressure wire 220 is translated longitudinally within the lumen 210, the stiffness of the TAVI guidewire 200 may vary. With the pressure wire 220 translated or withdrawn proximally, a distal portion of the TAVI guidewire 200 may become more flexible to facilitate tracking and navigation through tortuous vasculature. With the pressure wire 220 translated or extended distally, a distal portion of the TAVI guidewire 200 may become less flexible or more rigid to facilitate pushability and strong support.

Construction of the pressure wire 220 may be similar to the materials and/or methods described above with respect to a TAVI guidewire. In general, the pressure wire 220 may be a solid wire, although a tubular wire or shaft is also possible in some configurations. The proximal pressure sensor 222 and the distal pressure sensor 224 may be integrally formed with the pressure wire 220, or the proximal pressure sensor 222 and the distal pressure sensor 224 may be attached or otherwise assembled to the pressure wire 220, such as, but not limited to, by welding, soldering, brazing, crimping, friction fitting, adhesive bonding, mechanical interlocking, and the like.

While not expressly illustrated, in some embodiments, the lumen 210 and the pressure wire 220 may each include a cooperating orientation means such as a slot and key, a flattened side, or an orientation-defining shape (i.e., lobed, triangular, polygonal, etc.) which prevents rotation of the pressure wire 220 relative to the TAVI guidewire 200. In some embodiments, the orientation means may be beneficial in steering the TAVI guidewire 300 during navigation of tortuous vasculature and/or enhancing torque transmission from the proximal end 304 to the distal end 302.

In use, a distal end 202 of a tubular TAVI guidewire 200 having a plurality of apertures 260 disposed therein may be advanced percutaneously upstream within a patient's aorta 30 to a treatment site (i.e., a patient's aortic valve 10), The distal end 202 may be advanced through treatment site (i.e., the patient's aortic valve 10) into a patient's left ventricle 40 such that at least one of the plurality of apertures 260 is disposed distal of the treatment site (i.e., the patient's aortic valve 10) and at least one of the plurality of apertures 260 is disposed proximal of the treatment site (i.e., the patient's aortic valve 10) within a patient's aortic arch 20. In some embodiments, a pressure wire 220 having a distal pressure sensor 224 and a proximal pressure sensor 222 disposed thereon may be inserted into a proximal end 204 of the TAVI guidewire 200 and slidably advanced toward the distal end 202 within a lumen 210 of the TAVI guidewire 200. The distal pressure sensor 224 may be positioned adjacent the at least one of the plurality of apertures 260 disposed distal of the treatment site (i.e., the aortic valve 10), and the proximal pressure sensor 222 may be positioned adjacent the at least one of the plurality of apertures 260 disposed proximal of the treatment site (i.e., the patient's aortic valve 10). A patient's blood pressure within the left ventricle 40 and a patient's blood pressure within the aortic arch 20 may be measured using the distal pressure sensor 224 and the proximal pressure sensor 222, respectively. A blood pressure gradient across the treatment site (i.e., the patient's aortic valve 10), or a difference between the blood pressure distal of the treatment site (i.e., the patient's aortic valve 10), or within the left ventricle 40, and the blood pressure proximal of the treatment site (i.e., the patient's aortic valve 10), or within the aortic arch 20, may be measured, calculated, or otherwise determined. In some embodiments, the blood pressure gradient may be measured or determined prior to performing a TAVI procedure. In some embodiments, the blood pressure gradient may be spontaneously and/or continuously measured or determined during a TAVI procedure. In some embodiments, the blood pressure gradient may be measured or determined following a TAVI procedure. A method of measuring a patient's blood pressure gradient across a treatment site (i.e., a patient's aortic valve 10) may further include performing a TAVI procedure including advancing a TAVI device (not shown) distally over the TAVI guidewire 200 and percutaneously implanting a replacement aortic valve while maintaining the TAVI guidewire 200 and the pressure wire 220 in a generally fixed position within the treatment site (i.e., the patient's aortic valve 10), and/or the aorta 30, the aortic arch 20, and/or the left ventricle 40. In some embodiments, a method may further comprise withdrawing the pressure wire 220 from the TAVI guidewire 200 prior to removing the TAVI guidewire 200 from the treatment site (i.e., the patient's aortic valve 10). In some embodiments, a method may further comprise removing the TAVI guidewire 200 from the treatment site (i.e., the patient's aortic valve 10) and/or the patient.

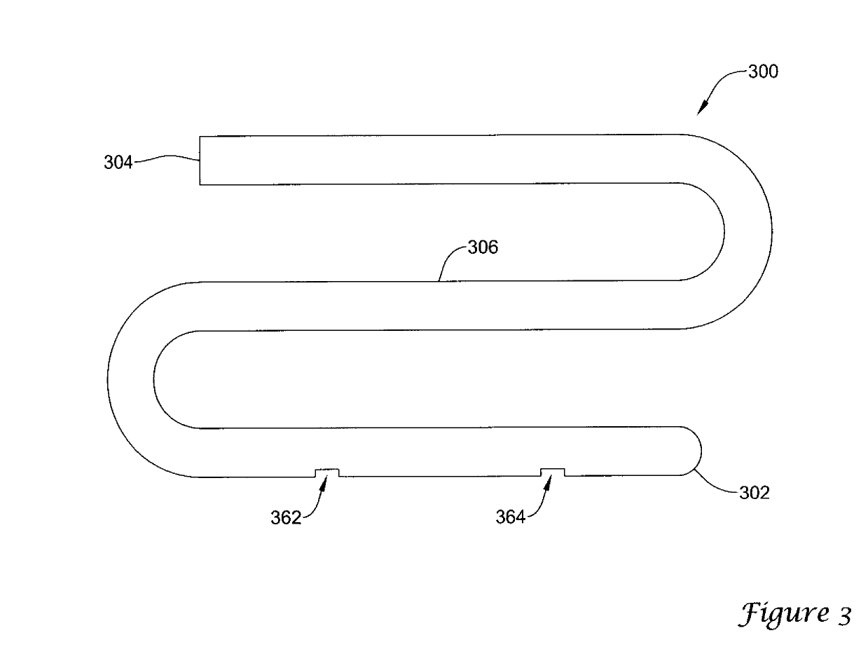

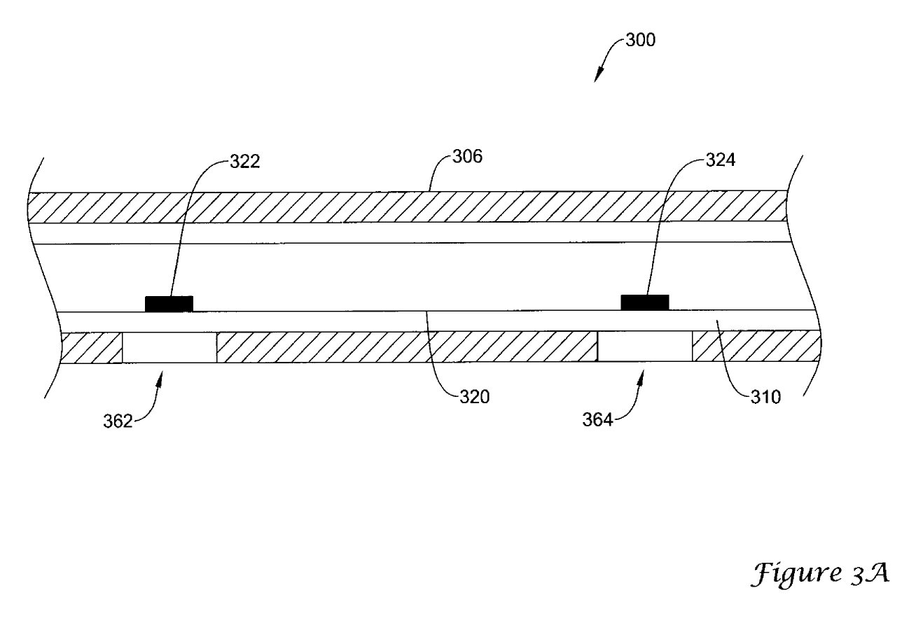

FIGS. 3 and 3A illustrate an example TAVI guidewire 300 having a lumen 310 disposed therein and a proximal aperture 362 and a distal aperture 364 through an outer wall 306 of the TAVI guidewire 300 disposed adjacent a distal end 302. In some embodiments, the proximal aperture 362 and the distal aperture 364 are longitudinally spaced apart along a length of the TAVI guidewire 300. Preferably, the proximal aperture 362 and the distal aperture 364 are longitudinally spaced apart far enough to permit placement on opposing sides (i.e., proximal and distal) of a treatment site (i.e., a patient's aortic valve 10). The TAVI guidewire 300 may include a pressure wire 320 slidably disposed within the lumen 310. The pressure wire 320 may include a proximal pressure sensor 322 and a distal pressure sensor 324 disposed thereon. The proximal pressure sensor 322 and the distal pressure sensor 324 may be longitudinally spaced apart along a length of the pressure wire 320. Preferably, the proximal pressure sensor 322 and the distal pressure sensor 324 are longitudinally spaced apart far enough to permit placement on opposing sides (i.e., proximal and distal) of a treatment site (i.e., a patient's aortic valve 10). In some embodiments, the proximal aperture 362 and the distal aperture 364 may be longitudinally spaced apart by a first distance. In some embodiments, the proximal pressure sensor 322 and the distal pressure sensor 324 may be longitudinally spaced apart by a second distance. In some embodiments, the first distance and the second distance may be substantially equal, while in other embodiments, the first distance and the second distance may be different. The proximal pressure sensor 322 and the distal pressure sensor 324 may be adapted and/or configured to measure a blood pressure gradient across a treatment site (i.e., a patient's aortic valve 10). In some embodiments, the pressure wire 320 may be a solid wire having an outer diameter of about 0.005 inch to about 0.025 inch, or in some embodiments, about 0.014 inch. The lumen 310 of the TAVI guidewire 300 may have a diameter slightly larger than the outer diameter of the pressure wire 320.

Additionally, as the pressure wire 320 is translated longitudinally within the lumen 310, the stiffness of the TAVI guidewire 300 may vary. With the pressure wire 320 translated or withdrawn proximally, a distal portion of the TAVI guidewire 300 may become more flexible to facilitate tracking and navigation through tortuous vasculature. With the pressure wire 320 translated or extended distally, a distal portion of the TAVI guidewire 300 may become less flexible or more rigid to facilitate pushability and strong support.

Construction of the pressure wire 320 may be similar to the materials and/or methods described above with respect to a TAVI guidewire. In general, the pressure wire 320 may be a solid wire, although a tubular wire or shaft is also possible in some configurations. The proximal pressure sensor 322 and the distal pressure sensor 324 may be integrally formed with the pressure wire 320, or the proximal pressure sensor 322 and the distal pressure sensor 324 may be attached or otherwise assembled to the pressure wire 320, such as, but not limited to, by welding, soldering, brazing, crimping, friction fitting, adhesive bonding, mechanical interlocking, and the like.

In some embodiments, the proximal aperture 362 and the distal aperture 364 may be aligned or face in a common direction, or toward a single side of the TAVI guidewire 300, as shown in FIGS. 3 and 3A. In some embodiments, the proximal aperture 362 and the distal aperture 364 may face in different directions, or toward different sides of the TAVI guidewire 300. While not expressly illustrated, in some embodiments, the lumen 310 and the pressure wire 320 may each include a cooperating orientation means such as a slot and key, a flattened side, or an orientation-defining shape (i.e., lobed, triangular, polygonal, etc.) which prevents rotation of the pressure wire 320 relative to the TAVI guidewire 300. In some embodiments, the orientation means may be beneficial in aligning the proximal pressure sensor 322 and the distal pressure sensor 324 with the proximal aperture 362 and the distal aperture 364, respectively. The orientation means may also be beneficial in steering the TAVI guidewire 300 during navigation of tortuous vasculature and/or enhancing torque transmission from the proximal end 304 to the distal end 302.

In use, a distal end 302 of a tubular TAVI guidewire 300 having a distal aperture 364 and a proximal aperture 362 disposed therein may be advanced percutaneously upstream within a patient's aorta 30 to a treatment site (i.e., a patient's aortic valve 10).

The distal end 302 may be advanced through the treatment site (i.e., the patient's aortic valve 10) into a patient's left ventricle 40 such that the distal aperture 364 is disposed distal of the treatment site (i.e., the patient's aortic valve 10) and the proximal aperture 362 is disposed proximal of the treatment site (i.e., the patient's aortic valve 10) within a patient's aortic arch 20. In some embodiments, a pressure wire 320 having a distal pressure sensor 324 and a proximal pressure sensor 322 disposed thereon may be inserted into a proximal end 304 of the TAVI guidewire 300 and slidably advanced toward the distal end 302 within a lumen 310 of the TAVI guidewire 300. The distal pressure sensor 324 may be positioned adjacent the distal aperture 364 disposed distal of the treatment site (i.e., the patient's aortic valve 10), and the proximal pressure sensor 322 may be positioned adjacent the proximal aperture 362 disposed proximal of the treatment site (i.e., the patient's aortic valve 10). A patient's blood pressure within the left ventricle 40 and a patient's blood pressure within the aortic arch 20 may be measured using the distal pressure sensor 324 and the proximal pressure sensor 322, respectively. A blood pressure gradient across the treatment site (i.e., the patient's aortic valve 10), or a difference between the blood pressure distal of the treatment site (i.e., the patient's aortic valve 10), or within the left ventricle 40, and the blood pressure proximal of the treatment site (i.e., the patient's aortic valve 10), or within the aortic arch 20, may be measured, calculated, or otherwise determined. In some embodiments, the blood pressure gradient may be measured or determined prior to performing a TAVI procedure. In some embodiments, the blood pressure gradient may be spontaneously and/or continuously measured or determined during a TAVI procedure. In some embodiments, the blood pressure gradient may be measured or determined following a TAVI procedure. A method of measuring a patient's blood pressure gradient across a treatment site (i.e., a patient's aortic valve 10) may further include performing a TAVI procedure including advancing a TAVI device (not shown) distally over the TAVI guidewire 300 and percutaneously implanting a replacement aortic valve while maintaining the TAVI guidewire 300 and the pressure wire 320 in a generally fixed position within the treatment site (i.e., the patient's aortic valve 10), and/or the aorta 30, the aortic arch 20, and/or the left ventricle 40. In some embodiments, a method may further comprise withdrawing the pressure wire 320 from the TAVI guidewire 300 prior to removing the TAVI guidewire 300 from the treatment site (i.e., the patient's aortic valve 10). In some embodiments, a method may further comprise removing the TAVI guidewire 300 from the treatment site (i.e., the patient's aortic valve) and/or the patient.

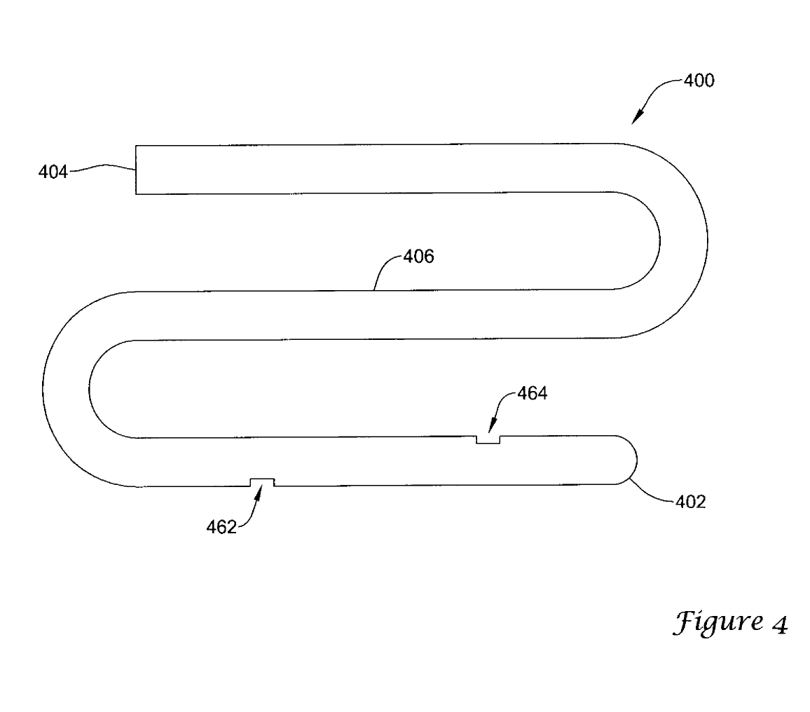

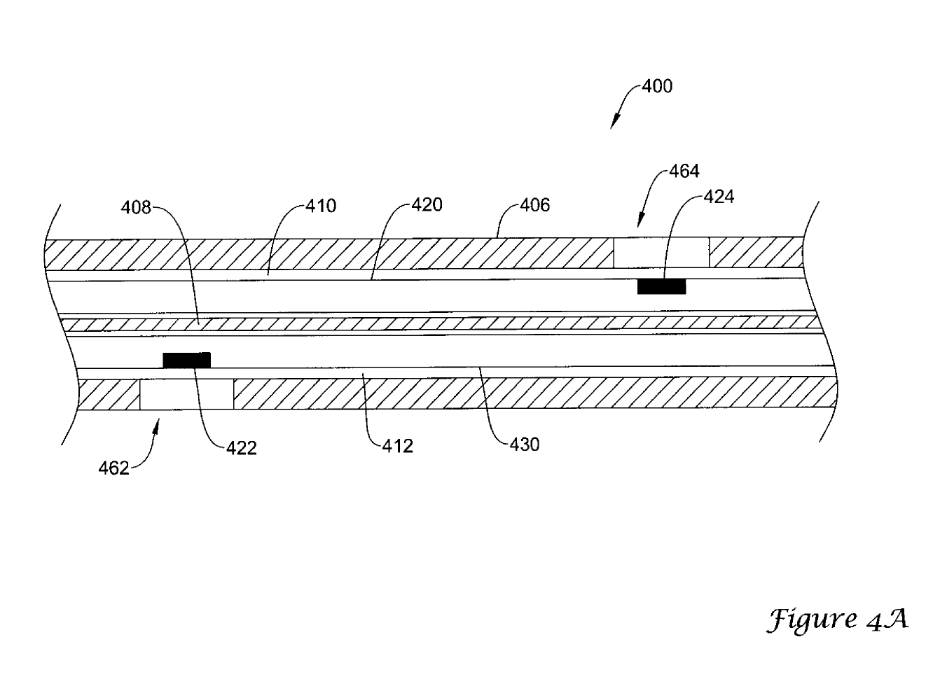

FIGS. 4 and 4A illustrate an example TAVI guidewire 400 having a first lumen 410 and a second lumen 412 disposed therein, and a proximal aperture 462 and a distal aperture 464 through an outer wall 406 of the TAVI guidewire 400 disposed adjacent a distal end 402. The first lumen 410 and the second lumen 412 may each be at least partially defined by an inner wall 408 of the TAVI guidewire 400 disposed between the first lumen 410 and the second lumen 412, and by a portion of the outer wall 406. In some embodiments, one or both of the first lumen 410 and the second lumen 412 may be round, hemispherical, or another suitable shape or cross-section. The inner wall 408 may extend from a proximal end 404 of the TAVI guidewire 400 to the distal end 402. In some embodiments, the inner wall 408 may be integrally formed with the TAVI guidewire 400. In some embodiments, one or both of the first lumen 410 and the second lumen 412 may terminate distal of the proximal end 404 at an opening or port (not shown) through the outer wall 406. In some embodiments, the proximal aperture 462 and the distal aperture 464 may be longitudinally spaced apart along a length of the TAVI guidewire 400. Preferably, the proximal aperture 462 and the distal aperture 464 are longitudinally spaced apart far enough to permit placement on opposing sides (i.e., proximal and distal) of a treatment site (i.e., a patient's aortic valve 10).

The TAVI guidewire 400 may include a first pressure wire 420 slidably disposed within the first lumen 410, and a second pressure wire 430 slidably disposed within the second lumen 412. The first pressure wire 420 may include a first pressure sensor 424, and the second pressure wire 430 may include a second pressure sensor 422 disposed thereon. The first pressure sensor 424 and the second pressure sensor 422 may be disposed at different longitudinal positions along the first pressure wire 420 and the second pressure wire 430, respectively. In other words, the first pressure sensor 424 may be positioned at a first distance proximal of a distal end of the first pressure wire 420, and the second pressure sensor 422 may be positioned at a second distance proximal of a distal end of the second pressure wire 430. In some embodiments, the first distance may be less than the second distance. In some embodiments, the second distance may be less than the first distance. The first pressure sensor 424 and the second pressure sensor 422 may be adapted and/or configured to cooperate with the distal aperture 464 and the proximal aperture 462, respectively, to measure a blood pressure gradient across a treatment site (i.e., a patient's aortic valve 10). In some embodiments, the first pressure wire 420 and/or the second pressure wire 430 may each be a solid wire having an outer diameter of about 0.005 inch to about 0.025 inch, or in some embodiments, about 0.014 inch. The first lumen 410 and/or the second lumen 412 of the TAVI guidewire 400 may each have a diameter slightly larger than the outer diameter of the first pressure wire 420 and/or the second pressure wire 430, respectively.

Additionally, as the first pressure wire 420 is translated longitudinally within the first lumen 410, and/or the second pressure wire 430 is translated longitudinally within the second lumen 412, the stiffness of the TAVI guidewire 400 may vary. With the first pressure wire 420 and/or the second pressure wire 430 translated or withdrawn proximally, a distal portion of the TAVI guidewire 400 may become more flexible to facilitate tracking and navigation through tortuous vasculature. With the first pressure wire 420 and/or the second pressure wire 430 translated or extended distally, a distal portion of the TAVI guidewire 400 may become less flexible or more rigid to facilitate pushability and strong support.

Construction of the first pressure wire 420 and/or the second pressure wire 430 may be similar to the materials and/or methods described above with respect to a TAVI guidewire. In general, the first pressure wire 420 and/or the second pressure wire 430 may each be a solid wire, although a tubular wire or shaft is also possible in some configurations. The first pressure sensor 424 and/or the second pressure sensor 422 may be integrally formed with the first pressure wire 420 and/or the second pressure wire 430, respectively, or the first pressure sensor 424 and/or the second pressure sensor 422 may be attached or otherwise assembled to the first pressure wire 420 and/or the second pressure wire 430, respectively, such as, but not limited to, by welding, soldering, brazing, crimping, friction fitting, adhesive bonding, mechanical interlocking, and the like.

In some embodiments, the proximal aperture 462 and the distal aperture 464 may be aligned or face in a common direction, or toward a single side of the TAVI guidewire 400. In some embodiments, the proximal aperture 462 and the distal aperture 464 may face in different directions, or toward different sides of the TAVI guidewire 400, as shown in FIGS. 4 and 4A. While not expressly illustrated, in some embodiments, the first lumen 410 and/or the second lumen 412, and the first pressure wire 420 and/or the second pressure wire 430, respectively, may each include a cooperating orientation means such as a slot and key, a flattened side, or an orientation-defining shape (i.e., lobed, triangular, polygonal, etc.) which prevents rotation of the first pressure wire 420 and/or the second pressure wire 430 relative to the TAVI guidewire 400. In some embodiments, the orientation means may be beneficial in aligning the first pressure sensor 424 and the second pressure sensor 422 with the distal aperture 464 and the proximal aperture 462, respectively. The orientation means may also be beneficial in steering the TAVI guidewire 400 during navigation of tortuous vasculature and/or enhancing torque transmission from the proximal end 404 to the distal end 402.

In use, a distal end 402 of a tubular TAVI guidewire 400 having a distal aperture 464 and a proximal aperture 462 disposed therein may be advanced percutaneously upstream within a patient's aorta 30 to a treatment site (i.e., a patient's aortic valve 10). The distal end 402 may be advanced through the treatment site (i.e., the patient's aortic valve 10) into a patient's left ventricle 40 such that the distal aperture 464 is disposed distal of the treatment site (i.e., the patient's aortic valve 10) and the proximal aperture 462 is disposed proximal of the treatment site (i.e., the patient's aortic valve 10) within a patient's aortic arch 20. In some embodiments, a first pressure wire 420 having a first pressure sensor 424 disposed thereon may be inserted into a proximal end 404 of the TAVI guidewire 400, and slidably advanced toward the distal end 402 within a first lumen 410 of the TAVI guidewire 400. In some embodiments, a second pressure wire 430 having a second pressure sensor 422 disposed thereon may be inserted into a proximal end 404 of the TAVI guidewire 400, and slidably advanced toward the distal end 402 within a second lumen 412 of the TAVI guidewire 400. The first pressure sensor 424 may be positioned adjacent the distal aperture 464 disposed distal of the treatment site (i.e., the patient's aortic valve 10), and the second pressure sensor 422 may be positioned adjacent the proximal aperture 462 disposed proximal of the treatment site (i.e., the patient's aortic valve 10). A patient's blood pressure within the left ventricle 40 and a patient's blood pressure within the aortic arch 20 may be measured using the first pressure sensor 424 and the second pressure sensor 422, respectively. A blood pressure gradient across the treatment site (i.e., the patient's aortic valve 10), or a difference between the blood pressure distal of the treatment site (i.e., the patient's aortic valve 10), or within the left ventricle 40, and the blood pressure proximal of the treatment site (i.e., the patient's aortic valve 10), or within the aortic arch 20, may be measured, calculated, or otherwise determined. In some embodiments, the blood pressure gradient may be measured or determined prior to performing a TAVI procedure. In some embodiments, the blood pressure gradient may be spontaneously and/or continuously measured or determined during a TAVI procedure. In some embodiments, the blood pressure gradient may be measured or determined following a TAVI procedure. A method of measuring a patients blood pressure gradient across a treatment site (i.e., a patient's aortic valve 10) may further include performing a TAVI procedure including advancing a TAVI device (not shown) distally over the TAVI guidewire 400 and percutaneously implanting a replacement aortic valve while maintaining the TAVI guidewire 400, the first pressure wire 420, and the second pressure wire 430 in a generally fixed position within the treatment site (i.e., the patient's aortic valve 10), and/or the aorta 30, the aortic arch 20, and/or the left ventricle 40. In some embodiments, a method may further comprise withdrawing the first pressure wire 420 and/or the second pressure wire 430 from the TAVI guidewire 400 prior to removing the TAVI guidewire 400 from the treatment site (i.e., the patient's aortic valve 10). In some embodiments, a method may further comprise removing the TAVI guidewire 400 from the treatment site (i.e., the patient's aortic valve 10) and/or the patient.

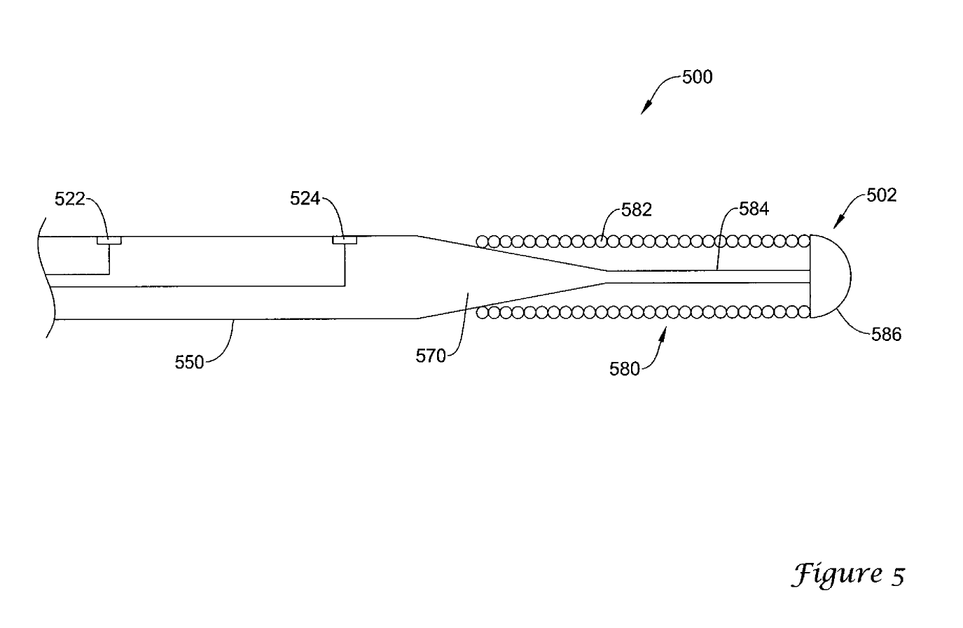

Refer now to FIG. 5, which illustrates a distal portion 550 of an example embodiment of a TAVI guidewire 500. The TAVI guidewire 500 may include similar structure(s) to that discussed above, with like reference numerals indicating similar structure. For example, the TAVI guidewire 500 may include a proximal pressure sensor 522 and a distal pressure sensor 524 mounted thereon.

The proximal pressure sensor 522 and the distal pressure sensor 524 may be longitudinally spaced apart along a length of the TAVI guidewire 500. Preferably, the proximal pressure sensor 522 and the distal pressure sensor 524 are longitudinally spaced apart far enough to permit placement on opposing sides (i.e., proximal and distal) of a treatment site (i.e., a patient's aortic valve 10). The proximal pressure sensor 522 and the distal pressure sensor 524 may be adapted and/or configured to measure a blood pressure gradient across a treatment site (i.e., a patient's aortic valve 10). In some embodiments, the TAVI guidewire 500 may be a generally solid wire having an outer diameter of about 0.010 inch to about 0.070 inch, or in some embodiments, about 0.035 inch.

Construction of the TAVI guidewire 500 may be similar to the materials and/or methods described above with respect to a TAVI guidewire. In general, the TAVI guidewire 500 may be a solid wire, although a tubular wire or shaft is also possible in some configurations. The proximal pressure sensor 522 and the distal pressure sensor 524 may be integrally formed with the TAVI guidewire 500, or the proximal pressure sensor 522 and the distal pressure sensor 524 may be attached or otherwise assembled to the TAVI guidewire 500, such as, but not limited to, by welding, soldering, brazing, crimping, friction fitting, adhesive bonding, mechanical interlocking, and the like.

In this embodiment, however, the TAVI guidewire 500 includes some additional/alternative structure in the distal portion 550 thereof, For example, while the TAVI guidewire 500 includes a distal end 502 as discussed above, the distal end 502 of the TAVI guidewire 500 may be formed as a distal tip 586. The TAVI guidewire 500 may include a structure 580 disposed adjacent to the distal tip 586. The structure 580 may include a flexible element 582 such as a coil, a spring, a helical winding, a polymer sheath, or other suitable flexible element, disposed over a shaping ribbon 584 or wire connecting the distal tip 586 to the distal portion 550. In some embodiments, the distal portion 550 may include a distally tapered section 570 connecting the distal portion 550 to the shaping ribbon 584 and/or the distal tip 586. In some embodiments, at least a portion of the flexible element 582 may be disposed about at least a portion of the distally tapered section 570. In some embodiments, the flexible element 582 may be fixedly attached to the distally tapered section 570 and/or the distal tip 586.

The structure 580 can be made from a variety of materials, including metals, alloys, plastics, or other suitable materials, for example, those discussed above. The cross-section of the structure 580, including the flexible member 582 and/or the shaping ribbon 584, can be of a variety of shapes, including round, oval, flat, ribbon-shaped, rectangular, square, or any other suitable shape or a combination thereof.

In the embodiment shown, the flexible element 582 is a helical coil. Such a coil may act to reinforce the shaping ribbon 584 and/or the distal tip 586 of the TAVI guidewire 500, and/or may act as a radiopaque marker, or both. The coil may be formed of or comprise wire or ribbon that has a solid cross-section, and may include any of a variety of cross-sectional shapes, including round, oval, flat, ribbon-shaped, or any other suitable shape or a combination thereof. The coil may be made of a variety of materials, including metals, alloys, plastics, or other suitable materials, including radiopaque materials, many of which were discussed above. Some examples of other suitable tip constructions and structures that can be used are disclosed in U.S. Pat. No. 6,918,882, and U.S. patent application Ser. No. 10/086,992 filed Feb. 28, 2002 (Pub. No. U.S. 2003/0069521), which are incorporated herein by reference.

In use, a distal end 502 of a TAVI guidewire 500 a distal pressure sensor 524 and a proximal pressure sensor 522 disposed thereon may be advanced percutaneously upstream within a patient's aorta 30 to a treatment site (i.e., a patient's aortic valve 10). The distal end 502 may be advanced through the treatment site (i.e., the patient's aortic valve 10) into a patient's left ventricle 40 such that the distal pressure sensor 524 is disposed distal of the treatment site (i.e., the patient's aortic valve 10) and the proximal pressure sensor 522 is disposed proximal of the treatment site (i.e., the patient's aortic valve 10) within a patient's aortic arch 20. A patient's blood pressure within the left ventricle 40 and a patient's blood pressure within the aortic arch 20 may be measured using the distal pressure sensor 524 and the proximal pressure sensor 522, respectively. A blood pressure gradient across the treatment site (i.e., the patient's aortic valve 10), or a difference between the blood pressure distal of the treatment site (i.e., the patient's aortic valve 10), or within the left ventricle 40, and the blood pressure proximal of the treatment site (i.e., the patient's aortic valve 10), or within the aortic arch 20, may be measured, calculated, or otherwise determined. In some embodiments, the blood pressure gradient may be measured or determined prior to performing a TAVI procedure. In some embodiments, the blood pressure gradient may be spontaneously and/or continuously measured or determined during a TAVI procedure. In some embodiments, the blood pressure gradient may be measured or determined following a TAVI procedure. A method of measuring a patient's blood pressure gradient across a treatment site (i.e., a patient's aortic valve 10) may further include performing a TAVI procedure including advancing a TAVI device (not shown) distally over the TAVI guidewire 500 and percutaneously implanting a replacement aortic valve while maintaining the TAVI guidewire 500 in a generally fixed position within the treatment site (i.e., the patient's aortic valve 10), and/or the aorta 30, the aortic arch 20, and/or the left ventricle 40. In some embodiments, a method may further comprise removing the TAVI guidewire 500 from the treatment site (i.e., the patient's aortic valve) and/or the patient.

It should be understood that although the above discussion was focused on a medical device and methods of use within the coronary vascular system of a patient, other embodiments of medical devices or methods in accordance with the invention can be adapted and configured for use in other parts of the anatomy of a patient. For example, devices and methods in accordance with the invention can be adapted for use in the digestive or gastrointestinal tract, such as in the mouth, throat, small and large intestine, colon, rectum, and the like. For another example, devices and methods can be adapted and configured for use within the respiratory tract, such as in the mouth, nose, throat, bronchial passages, nasal passages, lungs, and the like. Similarly, the medical devices described herein with respect to percutaneous deployment may be used in other types of surgical procedures as appropriate. For example, in some embodiments, the medical devices may be deployed in a non-percutaneous procedure, including an open heart procedure. Devices and methods in accordance with the invention can also be adapted and configured for other uses within the anatomy.

The present invention should not be considered limited to the particular examples described above, but rather should be understood to cover all aspects of the invention as fairly set out in the attached claims. Various modifications, equivalent processes, as well as numerous structures to which the present invention may be applicable will be readily apparent to those of skill in the art to which the present invention is directed upon review of the instant specification. It should be understood that this disclosure is, in many respects, only illustrative. Changes may be made in details, particularly in matters of shape, size, and arrangement of steps without exceeding the scope of the invention. The scope of the invention is, of course, defined in the language in which the appended claims are expressed.

We claim:

1. A guidewire system, comprising:
a tubular guidewire having an open proximal end, a closed distal end, and a length extending therebetween, the tubular guidewire including at least one lumen extending from the proximal end to the distal end; and
at least one pressure wire slidably disposed within the at least one lumen, the at least one pressure wire having a length and at least one pressure sensor disposed thereon;
wherein the tubular guidewire includes a plurality of apertures disposed through an outer wall of the tubular guidewire.

2. The guidewire system of claim 1, wherein the at least one pressure wire consists of a single pressure wire.

3. The guidewire system of claim 1, wherein the at least one pressure sensor comprises a proximal pressure sensor and a distal pressure sensor.

4. The guidewire system of claim 3, wherein the proximal pressure sensor is longitudinally spaced apart from the distal pressure sensor along the length of the at least one pressure wire.

5. The guidewire system of claim 3, wherein the proximal pressure sensor and the distal pressure sensor are positioned on the at least one pressure wire to face a common direction.

6. The guidewire system of claim 3, wherein the proximal pressure sensor and the distal pressure sensor are positioned on the at least one pressure wire to face different directions.

7. The guidewire system of claim 3, wherein the proximal pressure sensor and the distal pressure sensor are adapted to measure a blood pressure gradient across a target site.

8. The guidewire system of claim 1, wherein the tubular guidewire comprises a metallic hypotube.

9. The guidewire system of claim 1, wherein the plurality of apertures comprises a plurality of generally transverse slots cut into an outer wall of the tubular guidewire.

10. The guidewire system of claim 1, wherein the plurality of apertures comprises a proximal aperture and a distal aperture longitudinally spaced apart along the length of the tubular guidewire.

11. The guidewire system of claim 10, wherein the proximal aperture and the distal aperture are positioned in the tubular guidewire to face a common direction.

12. The guidewire system of claim 10, wherein the proximal aperture and the distal aperture are positioned in the tubular guidewire to face different directions.

13. The guidewire system of claim 1, wherein the at least one lumen includes a first lumen extending from the proximal end to the distal end and a second lumen extending from the proximal end to the distal end.

14. The guidewire system of claim 13, wherein the at least one pressure wire consists of a first pressure wire having a first pressure sensor disposed thereon and a second pressure wire having a second pressure sensor disposed thereon.

15. The guidewire system of claim 14, wherein the first pressure wire is slidably disposed within the first lumen and the second pressure wire is slidably disposed within the second lumen.

16. A method of measuring a blood pressure gradient across a treatment site, comprising:
obtaining a tubular guidewire system comprising:
a tubular guidewire having an open proximal end, a closed distal end, and a length extending therebetween, the tubular guidewire including at least one lumen extending from the proximal end to the distal end; and
a pressure wire slidably disposed within the at least one lumen, the pressure wire having a length, a proximal pressure sensor disposed thereon, and a distal pressure sensor disposed thereon;
wherein the tubular guidewire includes a plurality of apertures disposed through an outer wall of the tubular member;
advancing the distal end of the tubular guidewire upstream within a patient's vasculature to the treatment site;
positioning the distal end of the tubular guidewire distal of the treatment site such that at least one of the plurality of apertures is disposed distal of the treatment site and at least one of the plurality of apertures is disposed proximal of the treatment site;
translating the pressure wire longitudinally within the at least one lumen of the tubular guidewire;
positioning the pressure wire within the at least one lumen such that the distal pressure sensor is disposed distal of the treatment site and adjacent the at least one of the plurality of apertures disposed distal of the treatment site, and the proximal pressure sensor is disposed proximal of the treatment site and adjacent the at least one of the plurality of apertures disposed proximal of the treatment site; and
measuring a blood pressure gradient across the treatment site using the proximal pressure sensor and the distal pressure sensor.

17. The method of claim 16, further comprising:
advancing a transcatheter aortic valve implantation device distally over the tubular guidewire; and
performing a transcatheter aortic valve implantation procedure at the treatment site.

18. The method of claim 17, wherein the transcatheter aortic valve implantation procedure is performed while maintaining the tubular guidewire in a generally fixed position relative to the treatment site.

19. The method of claim 18, further comprising:
continuously measuring the blood pressure gradient during the transcatheter aortic valve implantation procedure.

20. The method of claim 16, wherein the tubular guidewire includes a distal portion having a stiffness;
wherein translating the pressure wire longitudinally within the at least one lumen of the tubular guidewire modifies the stiffness of the distal portion of the tubular guidewire; and
wherein the stiffness of the distal portion of the tubular guidewire is modified during the step of advancing the distal end of the tubular guidewire upstream within the patient's vasculature to the treatment site.
